(19) United States
(12) Patent Application Publication
(10) Pub. No.: US 2011/0105893 A1
(43) Pub. Date: May 5, 2011

(54) TISSUE TRACKING ASSEMBLY AND METHOD

(75) Inventors: Samuel Joseph Akins, Lawrence, MA (US); Ronald von Jako, Lawrence, MA (US)

(73) Assignee: GENERAL ELECTRIC COMPANY, Schenectady, NY (US)

(21) Appl. No.: 12/610,898

(22) Filed: Nov. 2, 2009

(51) Int. Cl. A61B 5/05

(52) U.S. Cl. 600/424

(57) ABSTRACT

A tissue tracking assembly and method is disclosed herewith. The tissue tracking assembly comprises: a flexible probe with a proximal end and distal end, the proximal end incorporates an electric connector and the distal end incorporates at least one trackable sensor. A delivery system is provided in association with the flexible probe to accurately embed the flexible probe within a tissue. The delivery system is configured to be attached to the flexible probe during insertion of probe within the tissue and detaches from the flexible probe after embedding the probe within the tissue.

### FIELD OF THE INVENTION

This invention relates generally to tissue tracking, and more particularly to, a tissue tracking probe and a delivery system for placing the tracking probe precisely within the tissue.

### BACKGROUND OF THE INVENTION

Probes containing electro magnetic sensors are used for tracking tissue and other devices within the body. Placing electromagnetically trackable devices in an organ, especially in a moving soft tissue, involves various challenges. The signal strength received from the tracking device must be sufficient for clinical interpretation and signal strength depends on various parameters including position and orientation of the trackable sensors. The position and orientation of tracking probe in the tissue is critical to represent the motion of the clinically relevant tissue. The probes described herein are flexible and may not be able to be delivered precisely within the body, without any support. Also, the probes might move relative to surrounding tissue motion or due to other external forces or organ motion.

The probe is used in conjunction with various existing tracking systems.

However to track effectively, it is essential that the probe is positioned accurately within the organ. Placing electromagnetically trackable devices or therapeutic devices in moving soft tissue is problematic because of the need for a dynamic reference that can track the tissue motion to properly register the image space with the tracker space. Tissue and organ displacements caused by respiratory, cardiovascular, gastrointestinal (GI) and cardiac motions need to be tracked in some applications to accurately place devices such as RF tumor ablation probes, hemostatic probes, embolization catheters, electrophysiology catheters, endovascular catheters, biopsy needles, or any other location-critical therapies using electromagnetic navigation.

Therefore, it is desirable to provide method and system for providing dynamic reference to track tissue motion accurately so that a therapeutic device can be registered with anatomical patient images. Thus there exists a need for an improved tissue tracking assembly capable of tracking moving soft tissues.

### SUMMARY OF THE INVENTION

The above-mentioned shortcomings, disadvantages and problems are addressed herein which will be understood by reading and understanding the following specification.

One embodiment of the present invention provides a tissue tracking assembly. The assembly comprises: a flexible probe with a proximal end and a distal end, the proximal end incorporates an electric connector and the distal end incorporates a sensor assembly; and a delivery system associated with the flexible probe, adapted for embedding the flexible probe within a tissue; wherein the delivery system is configured to be attached to the flexible probe during insertion of probe within the tissue and then detached from the flexible probe after embedding the probe within the tissue.

In another embodiment, a tissue tracking assembly is provided. The assembly comprises: a flexible probe with a proximal end and a distal end, the proximal end incorporates an electric connector and the distal end incorporates at least one trackable sensor; a delivery system associated with the flexible probe, adapted for embedding the flexible probe within the tissue. The delivery system comprises: a needle assembly having an open first end and an open second end with a lumen extending there between, the first end comprises a pointed tip and the second end comprises a detachable clamping mechanism configured to be attached to the flexible probe while inserting the probe within the tissue and detached from the flexible probe after embedding the probe within the tissue; and a reinforcing tube associated with the needle assembly for supporting the flexible probe during insertion of the flexible probe within the tissue.

In yet another embodiment, a method of tissue tracking using a tissue tracking assembly having a flexible probe with a distal end having at least one trackable sensor and a proximal end and a delivery system having a needle assembly with a needle, a clamping mechanism and a reinforcing tube attachable to the needle assembly is disclosed. The method comprises: attaching clamping mechanism to the needle assembly; inserting reinforcing tube into an open lumen of the needle assembly; inserting proximal end of the flexible probe axially into the reinforcing tube; engaging distal end of the reinforcing tube with the distal end of the probe; and clamping the clamping mechanism to the flexible probe for securing the flexible probe to the delivery system.

Various other features, objects, and advantages of the invention will be made apparent to those skilled in the art from the accompanying drawings and detailed description thereof.

### BRIEF DESCRIPTION OF THE DRAWINGS

FIG. 1 is a block diagram of a tissue tracking assembly as described in an embodiment of the invention;

FIG. 2 illustrates a diagrammatic representation of a tissue tracking assembly as described in an embodiment of the invention;

FIG. 3 illustrates a diagrammatic representation of a tissue tracking assembly along with an ablator as described in an embodiment of the invention;

FIG. 4 illustrates a diagrammatic representation of a tissue tracking assembly along with depth markers as described in an embodiment of the invention;

FIG. 5 illustrates a diagrammatic representation of a tissue tracking assembly along with drain channel as described in an embodiment of the invention;

FIG. 6 illustrates a diagrammatic representation of a tissue tracking assembly along with anchoring mechanism as described in an embodiment of the invention;

FIG. 7 is a flowchart illustrating a method of tracking tissues as described in an embodiment of the invention; and

FIG. 8 is a detailed flowchart illustrating a method of tracking tissues as described in an embodiment of the invention.

### DETAILED DESCRIPTION OF THE INVENTION

In the following detailed description, reference is made to the accompanying drawings that form a part hereof, and in which is shown by way of illustration specific embodiments that may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken as limiting the scope of the invention. To the extent that the figures illustrate diagrams of the functional blocks of various embodiments, the functional blocks are not necessarily indicative of the division between hardware circuitry. Thus, for example, one or more of the functional blocks may be implemented in as single unit. Similarly, the programs may be stand alone programs, may be incorporated as subroutines in an operating system, may be functions in an installed software package, and the like. It should be understood that the various embodiments are not limited to the arrangements and instrumentality shown in the drawings.

Embodiments of the present invention assist in tracking tissues using electromagnetic tracking devices. To achieve this, an exemplary embodiment of the present invention utilizes a method of providing a dynamic reference for registering the image. The dynamic reference is obtained by placing the tracking assembly accurately within the tissue and tracking the tissue accurately. A flexible tube having electromagnetically trackable sensors is embedded within the tissue with the assistance of a delivery system. The tracked tissue motion is used in placing a therapeutic device accurately within the tissue.

In an embodiment, a flexible probe with a sensor assembly is placed within the tissue using a delivery system having a needle assembly and a reinforcing tube. The invention is particularly useful in analyzing visceral tissue motion and placing a therapeutic device precisely within the tissue based on the tracked tissue motion.

In various embodiments, the therapeutic devices could include RF tumor ablation probes, hemostatic probes, embolization catheters, electrophysiology catheters, endovascular catheters, biopsy needles, or any other location-critical therapies using electromagnetic navigation, however need not be limited to these.

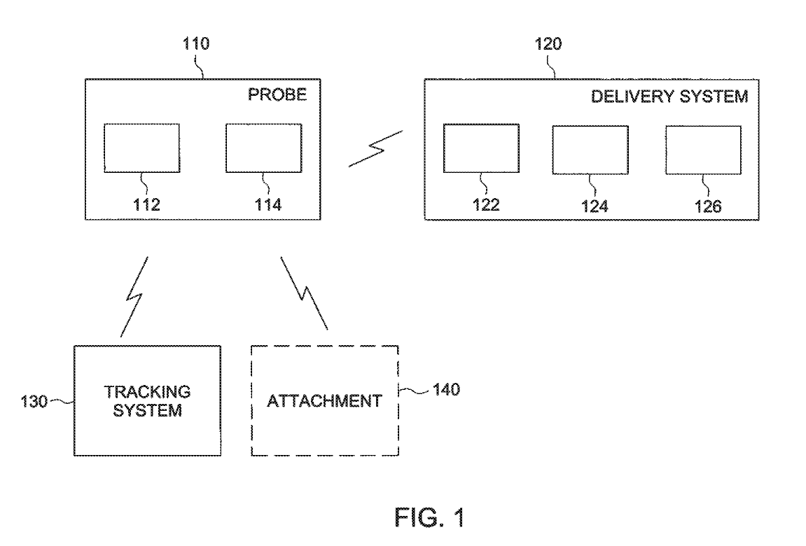

FIG. 1 is a block diagram of a tissue tracking assembly as described in an embodiment of the invention. The assembly comprises a flexible probe configured to track the tissue. The flexible probe 110 has a distal end and a proximal end. The distal end is associated with a sensor assembly 112 having at least one micro-coil or trackable sensor. The proximal end incorporates a connector 114. The sensor assembly 112 is connected to the connector 114 through electrical wiring such as coax cable, electrical wire or twisted pairs of wires. The sensor assembly 112 could also include different sensors capable of tracking tissues. The configuration of sensor assembly 112 might depend on the nature of the tracking, organ, tissue etc. The flexible probe 110 is flexible enough to follow tissue motion and has sufficient surface friction to prevent motion relative to the tissue to accurately track the tissue motion and is strong enough to allow insertion and removal of the probe 110.

In an embodiment, a delivery system 120 is provided to embed the probe 110 within the tissue. Since the flexible probe 110 is flexible, the delivery system 120 assists the probe 110 in placing the same accurately within the tissue. The delivery system 120 includes a needle assembly 122, a clamping mechanism 124 and a reinforcing tube 126. The needle assembly 122 includes an open first end and a second end connected via an open lumen. The open lumen accommodates the probe while inserting the probe 110 into the tissue. The open lumen can slide over the probe temporarily to add rigidity to the probe 110 for tissue insertion. First end of the needle assembly is a pointed tip, which is used in making an insertion into the human body, through which the probe 110 is inserted into the body. At other end of the lumen, or the second end of the needle assembly 122, the clamping mechanism 124 such as a touhy borst is provided. The clamping mechanism 124 is capable of clamping and sealing the probe while the probe 110 is under tension. The delivery system 120 further includes a reinforcing tube 126. The reinforcing tube 126 could be a part of the needle assembly 122. The reinforcing tube 126 is configured to support the probe 110 axially and prevent buckling while inserting the probe 110 into the tissue. In an embodiment, the sensor assembly 112 includes a locking mechanism configured to mate the probe 110 with the reinforcing tube 126. The locking feature for the rigid reinforcing tube 126 and the cylindrical shape will act as an anchoring mechanism preventing axial displacement of the sensor assembly with respect to the tissue or organ movement. The reinforcing tube 126 could be attached to the clamping mechanism 124 or could be integrated with the wall of the needle assembly 122.

While inserting the probe 110 into the tissue, the clamping mechanism 124 is attached to the reinforcing tube 126. The probe 110 is inserted into the reinforcing tube 126 and the clamping mechanism 124 clamps and seals the probe 110. Once the probe 110 is in the tissue, the clamping mechanism 124 is released and the reinforcing tube 126 along with the clamping mechanism 124 retracts leaving the probe 110 within the tissue. The probe 110 is placed in the tissue and it is ready to be tracked and used to determine tissue motion relative to a therapeutic device. At the end of the procedure, the probe may be removed from the tissue by pulling the tracking assembly out by hand pressure or by advancing delivery system over probes and retracting probes and delivery system from the tissue.

In various embodiments, the sensing assembly 112 could include, any tracking devices, including electromagnetic trackable sensors. The electromagnetic trackable sensor may interact with a tracking system 130 including various surgical navigation systems. The surgical navigation systems track the precise location of surgical instruments in relation to multidimensional images of a patient's anatomy. Additionally, surgical navigation systems use visualization instruments to provide the surgeon with co-registered views of these surgical instruments with the patient's anatomy. The surgical navigation system determines the position and/or orientation of the trackable sensor within a surgical instrument e.g., a guidewire or a catheter, needle or probes and conveys this location to a user. The position and orientation information can be conveyed by virtually superimposing a graphic representation of a portion of the surgical instrument onto a patient image. The surgical instrument can be viewed in real-time or near real-time as it passes through the patient. Accordingly, the user receives visual feedback to help navigate or guide the surgical instrument to the target site. Thus the accurate positioning of sensor assembly 112 helps in surgical navigation.

In an embodiment, an attachment 140 could be connected to the needle assembly for performing a secondary function. The attachment 140 could include an ablator, drain channel, depth markers, anchoring mechanisms etc, but need not be limited to these examples. The attachment 140 may be use wired or wireless technology to interact with the needle assembly 122 or with the electrical/energy connectors

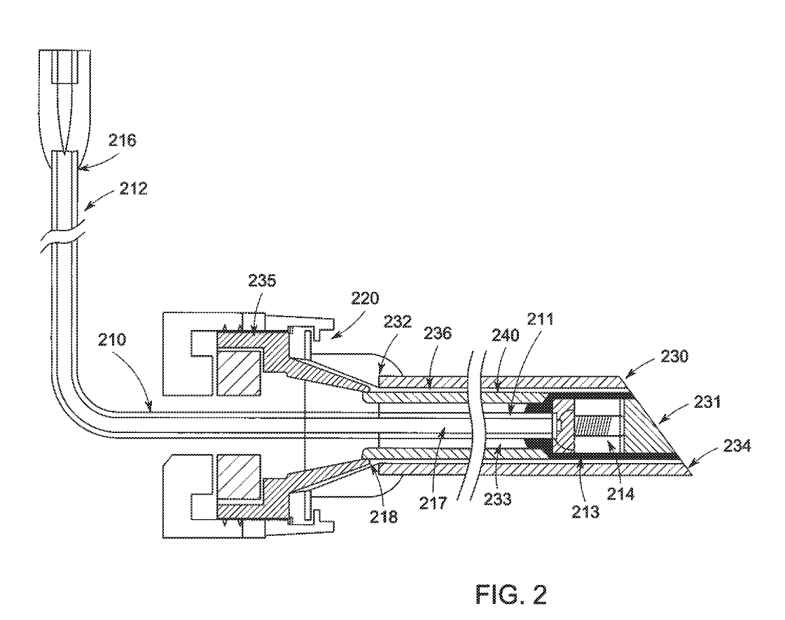

FIG. 2 illustrates a diagrammatic representation of a tissue tracking assembly as described in an embodiment of the invention. The tissue tracking assembly comprises a flexible probe 210 for tracking the tissues and a delivery system 220 for delivering and embedding the flexible probe within the tissue for tracking. The flexible probe includes a distal end 211 and a proximal end 212. The distal end 211 incorporates at least one sensor assembly 213 capable of tracking the tissues. The sensor assembly 213 could include at least one sensor 214 and optionally an interlocking mechanism 218. The proximal end 212 of the probe 210 is connected to a connector 216. The sensors 214 are connected to the connector 216 electronically, in an example through a cable 217. In an embodiment, the probe 210 could be enclosed in a flexible enclosure or the probe 210 itself could be made of flexible material.

The delivery system 220 further comprises a needle assembly 230 and a reinforcing tube 240. In an embodiment, the reinforcing tube 240 could be integrated as a part of the needle assembly 230. The needle assembly 230 comprises a first end 231 and a second end 232. Both ends are open and connected via an open lumen 233. The first end 231 includes a pointed tip 234 configured to make an insertion into the body, providing an insertion path for the probe 210. The second end 232 includes a clamping mechanism 235 configured to hold and seal the probe 210 while the probe 210 is under tension.

In an embodiment, the reinforcing tube 240 is a rigid tube is configured to support the flexible probe 210 during insertion of the probe 210 to the body. The reinforcing tube 240 could be associated with needle wall 236 or to the claiming mechanism 235.

The flexible probe 210 is being inserted into the reinforcing tube 240 initially. The reinforcing tube 240 supports the flexible probe 210 axially. The sensor assembly 213 of the probe 210 includes an interlocking feature 218, which mated with the rigid reinforcing tube 240. The clamping mechanism 235 is attached to the needle wall or to the reinforcing tube 240. Once the probe 210 is inserted into the reinforcing tube 240, the clamping mechanism 235 clamps and seals the probe 210 while the probe 210 is inserted into the reinforcing tube 240. The clamping mechanism 240 is attached to the needle assembly 230 while the probe 210 is being inserted into the body. Once the probe 210 reaches the desired organ or desired area that needs to be tracked, the clamping mechanism 235 is released and the needle assembly 230 along with the reinforcing tube 240 is retracted leaving the probe 210 within the tissue. In an embodiment, the clamping mechanism 235 is a touhy borst.

In an embodiment, the first end 231 of the needle assembly 230 may have an attachment configured to perform a secondary function. This attachment is optional and depends on the desired functionality or application of the probe. Various examples of attachments along with their functionality and arrangement are described in the following figures. However, the possible attachments need not be limited to these.

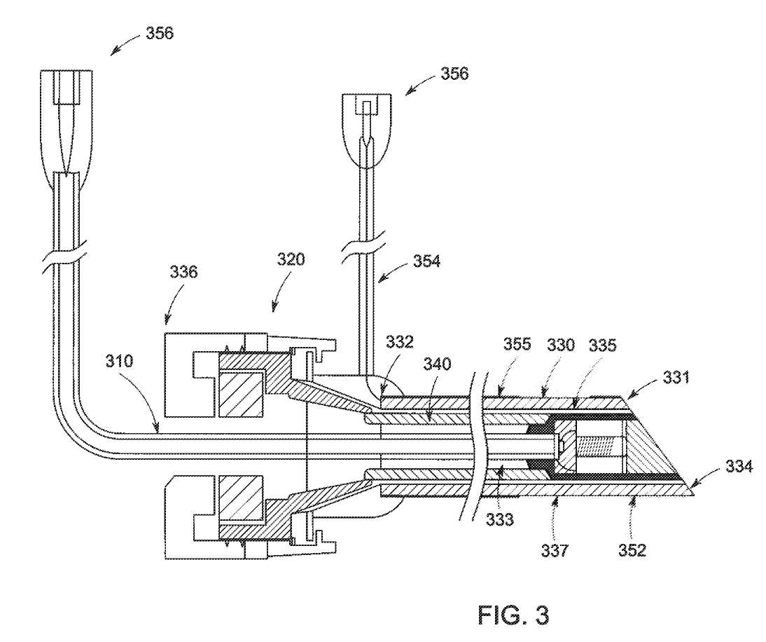

FIG. 3 illustrates a diagrammatic representation of a tissue tracking assembly along with an ablator as described in an embodiment of the invention. The tissue assembly could include a flexible probe 310 and a delivery system 320 for delivering the probe into the tissue. The delivery system 320 could include a needle assembly 330 and reinforcing tube 340. The needle assembly 330 includes a first end 331 and a second end 332. Both ends are open and connected via an open lumen 333. The first end 331 includes a pointed tip 334 configured to make an insertion into the body providing an insertion path for the probe 310. The needle assembly 330 could have a needle wall 335, needle hub 336 and a needle shaft 337.

In an embodiment, the needle assembly 330 can be attached to an ablator or electro surgery generators. This could be a low-energy bipolar option provided at the pointed tip 334 of the needle assembly 330. The energy can be delivered through an exposed electrode 352 placed on the outer circumference of the needle to coagulate blood to prevent bleeding during and after introducer insertion. An energy wire 354 can be routed through the needle hub 336 and attached to the needle shaft 337. The energy wire 354 may be connected to a connector 356. Alternately, the needle shaft 337 may be used as the electrode, with insulating material 355 covering a portion of the needle surface area to limit the area of ablation.

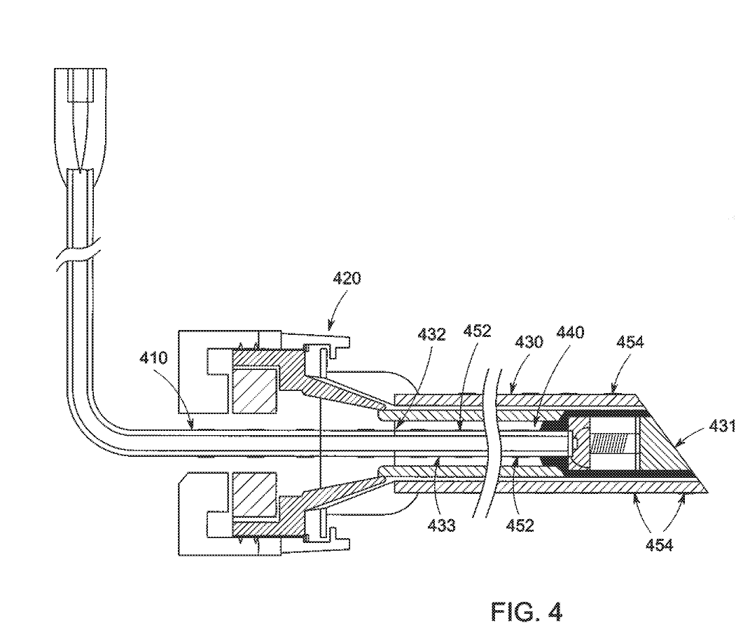

FIG. 4 illustrates a diagrammatic representation of a tissue tracking assembly along with depth markers as described in an embodiment of the invention. The tissue assembly could include a flexible probe 410 and a delivery system 420 for delivering the probe into the tissue. The delivery system 420 could include a needle assembly 430 and reinforcing tube 440. The needle assembly 430 includes a first end 431 and a second end 432. Both ends are open and connected via an open lumen 433. In an embodiment, one or more depth markers may be marked along probe shaft 452 or needle shaft 454 or along both. The depth markers 452, 454 will assist in identifying depth of the probe 410 or needle assembly 430 in the body.

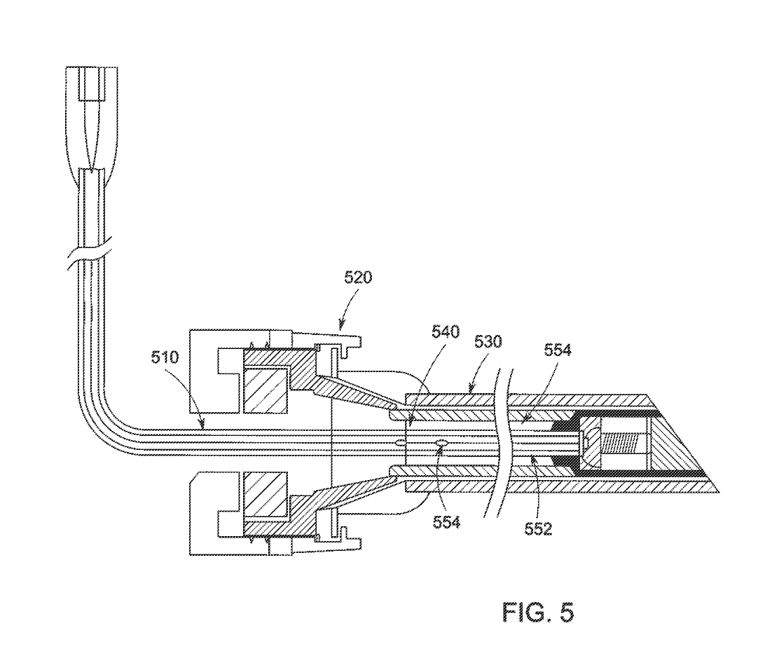

FIG. 5 illustrates a diagrammatic representation of a tissue tracking assembly along with drain channel as described in an embodiment of the invention. The tissue assembly could include a flexible probe 510 and a delivery system 520 for delivering the probe into the tissue. The delivery system 520 could include a needle assembly 530 and reinforcing tube 540. In an embodiment, the probe 510 could include at least one drain channel 552 to allow fluid drainage. Fluid drainage can be used as an indicator of internal bleeding or other visual leakage indicator. The lumen can have one or more holes 554 in its wall along the distal length to allow for fluid ingress. The drain channel 552 used could vary based on the application.

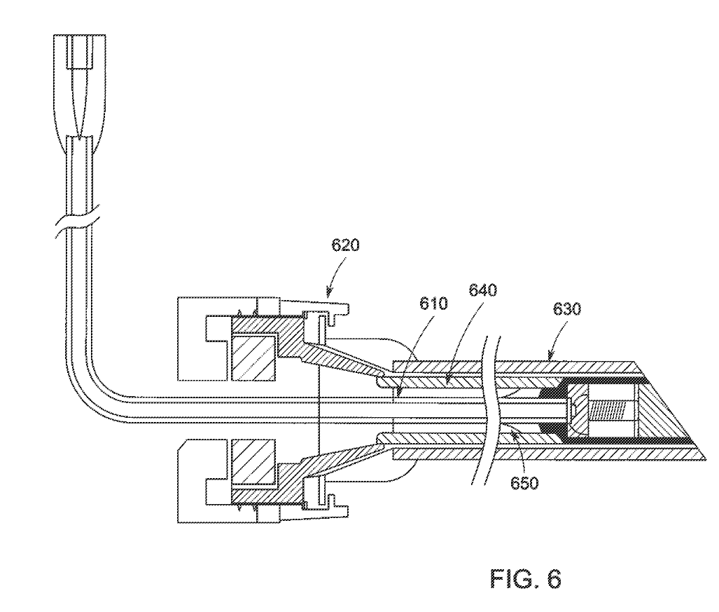

FIG. 6 illustrates a diagrammatic representation of a tissue tracking assembly along with an anchoring mechanism as described in an embodiment of the invention. The tissue assembly could include a flexible probe 610 and a delivery system 620 for delivering the probe into the tissue. The delivery system 620 could include a needle assembly 630 and reinforcing tube 640.

In an embodiment, additional anchoring mechanisms 650 along the probe length are provided to further prevent probe motion relative to the tissue. These anchoring mechanism 650 can be additional indents/bumps along the probe length or protruding anchor wings that spring open when the probe is released from the delivery system 620 and can be retracted/collapsed when removing the probe 610 from the tissue. The figure shows wings in collapsed or retracted position.

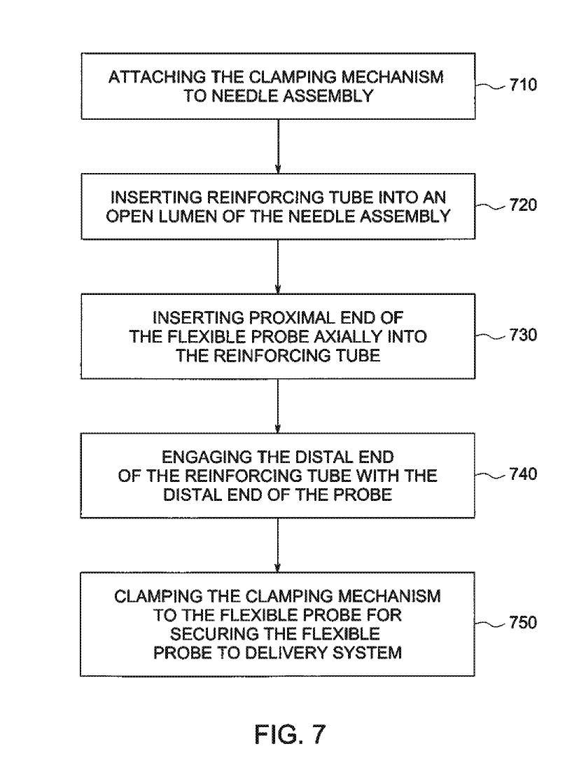

FIG. 7 is a flowchart illustrating a method of tracking tissues as described in an embodiment of the invention. For tissue tracking a tissue tracking assembly as described earlier in one or more of the embodiments is provided. In an embodiment, the tissue is tracked using a tracking assembly having a flexible probe with a proximal end having at least one trackable sensor and a distal end and a delivery system having a needle assembly with a needle, clamping mechanism attachable to the needle assembly and a reinforcing tube. At step 710, the claiming mechanism is attached to the needle assembly. The clamping mechanism could be a part of the reinforcing tube or needle assembly. At step 720, the reinforcing tube is inserted into the open lumen of the needle assembly. The reinforcing tube supports the flexible probe axially while inserting the probe into the tissue. At step 730, proximal end of the flexible probe is inserted into the reinforcing probe. The reinforcing tube provides additional support to the flexible probe. At step 740, the distal end of the reinforcing tube is engaged with the distal end of the probe. At step 750, the clamping mechanism associated with the reinforcing tube or the needle assembly clamps and seals the flexible probe. The clamping mechanism prevents probe from releasing from the delivery system during probe insertion into the tissue. Thus the flexible probe is ready to be inserted into the tissue with the help of the delivery system.

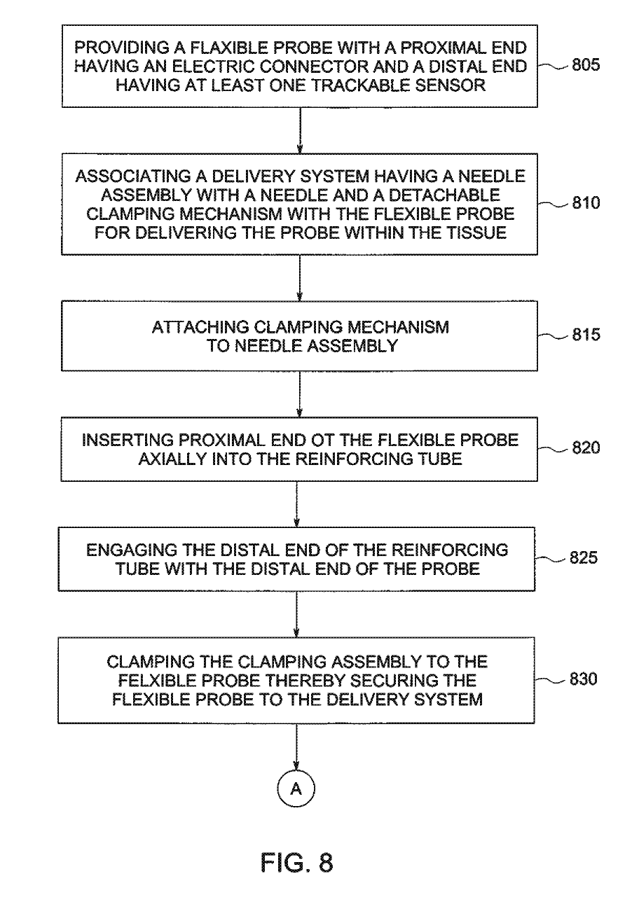

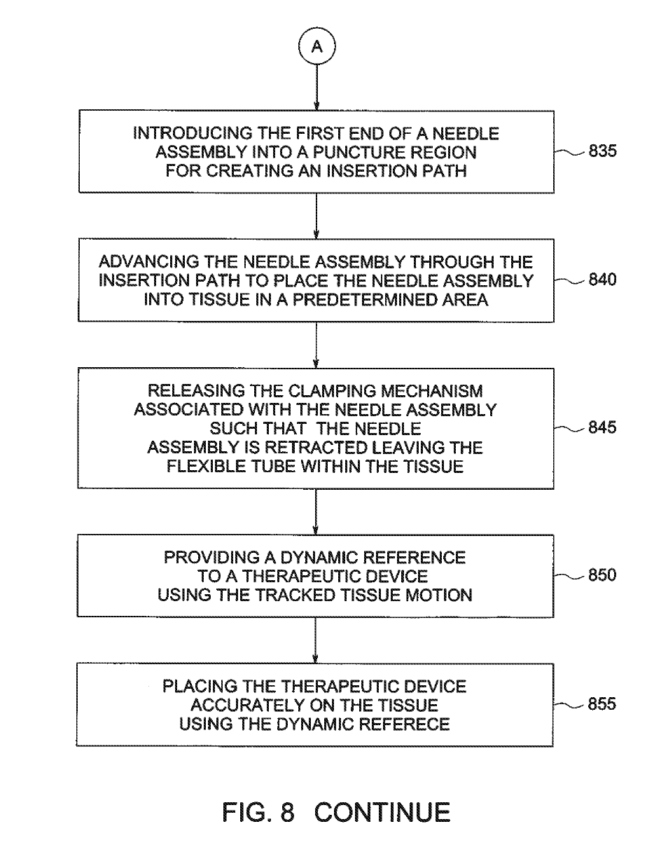

FIG. 8 is a detailed flowchart illustrating a detailed method of tracking tissues as described in an embodiment of the invention. At step 805, for tracking tissues a tracking assembly having a flexible probe with a distal end having at least one trackable sensor and a proximal end having an electric connector is provided. At step 810, associating the flexible probe with a delivery system having a needle assembly with a needle, and a clamping mechanism and a reinforcing tube attachable to the needle assembly. At step 815, the claiming mechanism is attached to the needle assembly. The clamping mechanism could be a part of the reinforcing tube or the needle assembly. At step 820, proximal end of the flexible probe is inserted into the reinforcing probe. The reinforcing tube provides additional support to the flexible probe. At step 825, the distal end of the reinforcing tube is engaged with the proximal end of the probe. At step 830, the clamping mechanism associated with the reinforcing tube or the needle assembly clamps and seals the flexible probe. The clamping mechanism prevents the probe buckling within the needle assembly while inserting the probe into the tissue. At step 835 the first end of the needle assembly is introduced into a puncture region for creating an insertion path and the needle assembly is advanced through the insertion path to place the needle assembly into the tissue in a predetermined area as at step 840. At step 845, the clamping mechanism is released such that the needle assembly is retracted leaving the flexible probe within the tissue. At step 850, a dynamic reference is obtained using the tracking of tissue. The relative position of a tissue to a therapeutic device is identified using the tracked tissue motion and thereby a dynamic reference is obtained. At step 855, the therapeutic device is placed accurately within the tissue with reference to the obtained dynamic reference.

The advantages of the invention include providing a device capable of facilitating the tracking of soft tissue motion to allow for more accurate, effective and efficient placement of therapeutic devices. The soft tissue motion is tracked using electromagnetic tracking systems. The tissue motion is then used to determine relative position of the tissue to a device used in applying therapy to the surrounding tissue.

The invention further provides a disposable accessory for an electromagnetic tracking system that facilitates least invasive methods of tracking various soft tissue procedures that benefit from improved visualization and anatomical orientation of a specific intracorporal apparatus.

The above-description of the embodiments of the methods and systems has the technical effect of tracking tissues.

Thus various embodiments of the invention describe a method and system for facilitating automated navigation in a healthcare environment.

As used herein, an element or step recited in the singular and proceeded with the word “a” or “an” should be understood as not excluding plural elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising” or “having” an element or a plurality of elements having a particular property may include additional such elements not having that property.

Exemplary embodiments are described above in detail. The assemblies and methods are not limited to the specific embodiments described herein, but rather, components of each assembly and/or method may be utilized independently and separately from other components described herein. Further the steps involved in the workflow need not follow the sequence in which there are illustrated in figures and all the steps in the work flow need not be performed necessarily to complete the method.

While the invention has been described with reference to preferred embodiments, those skilled in the art will appreciate that certain substitutions, alterations and omissions may be made to the embodiments without departing from the spirit of the invention. Accordingly, the foregoing description is meant to be exemplary only, and should not limit the scope of the invention as set forth in the following claims.

We claim:

1. A tissue tracking assembly comprising:
a flexible probe with a proximal end and a distal end, the proximal end incorporates an electric connector and the distal end incorporates a sensor assembly; and a delivery system associated with the flexible probe, adapted for embedding the flexible probe within a tissue;
wherein the delivery system is configured to be attached to the flexible probe during insertion of the flexible probe within the tissue and to be detached from the flexible probe after embedding the flexible probe within the tissue.

2. The assembly as claimed in claim 1, wherein the delivery system includes a needle assembly, a reinforcing tube and a clamping mechanism.

3. The assembly as claimed in claim 1, wherein the delivery system includes an attachment for performing a secondary function, the attachment including ablator, depth marker and anchoring mechanisms.

4. A tissue tracking assembly comprising:
a flexible probe with a proximal end and a distal end, the proximal end incorporates an electric connector and the distal end incorporates at least one trackable sensor;
a delivery system associated with the flexible probe, adapted for embedding the flexible probe within tissue, comprising:
a needle assembly having an open first end and an open second end with a lumen extending there between, the first end comprises a pointed tip and the second end comprises a detachable clamping mechanism configured to be attached to the flexible probe while inserting the flexible probe within the tissue and to be detached from the flexible probe after embedding the flexible probe within the tissue; and
a reinforcing tube associated with the needle assembly for supporting the flexible probe during insertion of the flexible probe within the tissue.

5. The assembly as claimed in claim 1, wherein the flexible probe includes at least one trackable sensor embedded in a flexible enclosure for tracking tissue motion.

6. The assembly as claimed in claim 1, wherein the flexible probe has surface friction sufficient to prevent relative motion of the flexible probe with respect to the tissue, and the flexible probe has strength sufficient to allow flexible probe insertion and removal.

7. The assembly as claimed in claim 1, wherein the trackable sensor includes a locking mechanism for locking the trackable sensor to the reinforcing tube.

8. The assembly as claimed in claim 1, wherein the open lumen is configured to accommodate the flexible probe while embedding the probe within the tissue.

9. The assembly as claimed in claim 1, wherein the reinforcing tube is configured to axially support the flexible probe while embedding the probe within the tissue.

10. The assembly as claimed in claim 1, wherein reinforcing tube is associated with needle wall.

11. The assembly as claimed in claim 1, wherein reinforcing tube is associated with the clamping mechanism.

12. The assembly as claimed in claim 1, wherein the clamping mechanism is configured to clamp and seal the flexible probe while probe is being inserted into the reinforcing tube.

13. The assembly as claimed in claim 1, wherein the probe includes a draining channel along the probe.

14. The assembly as claimed in claim 1, wherein the first end of the needle assembly includes an attachment for performing a secondary function.

15. The assembly as claimed in claim 14, wherein the attachment includes an ablator having a low energy bipolar option, comprising an electrode, electrical connector, and an energy wire connecting the electrode and connector.

16. The assembly as claimed in claim 14, wherein the attachment includes depth markers along the needle assembly and the flexible probe.

17. The assembly as claimed in claim 14, wherein the attachment includes anchoring mechanism to limit probe motion relative to tissue.

18. A method of tissue tracking using a tissue tracking assembly having a flexible probe with a proximal end and a distal end having at least one trackable sensor and a delivery system having a needle assembly with a needle, clamping mechanism and a reinforcing tube attachable to the needle assembly, the method comprising;
attaching the clamping mechanism to the needle assembly;
inserting the reinforcing tube into an open lumen of the needle assembly;
inserting the proximal end of the flexible probe axially into the reinforcing tube;
engaging the distal end of the reinforcing tube with the proximal end of the probe; and
clamping the clamping mechanism to the flexible probe for securing the flexible probe to the delivery system.

19. The method as claimed in claim 18 further comprises:
introducing the first end of a needle assembly into a puncture region for creating an insertion path;
advancing the needle assembly through the insertion path to place the needle assembly into tissue in a predetermined area; and
releasing the clamping mechanism associated with the needle assembly such that the needle assembly is retracted leaving the flexible probe within the tissue

20. The method as claimed in claim 18 further comprises: providing a dynamic reference to a therapeutic device using the tracked tissue motion and placing the therapeutic device accurately within the tissue.
